(19) United States
(12) Patent Application Publication
(10) Pub. No.: US 2024/0252691 A1
(43) Pub. Date: Aug. 1, 2024

(54) MAGNETIC NANOPARTICLES FOR IMAGE DIAGNOSIS AND CONTRAST MEDIUM FOR IMAGE DIAGNOSIS

(71) Applicant: OSAKA UNIVERSITY, Suita-shi, Osaka (JP)

(72) Inventors: Satoshi SEINO, Suita-shi, Osaka (JP); Hiroto IKEHATA, Suita-shi, Osaka (JP); Takami TOMIYAMA, Osaka-shi, Osaka (JP); Tomohiro UMEDA, Osaka-shi, Osaka (JP); Akihiro IZAWA, Koto-ku, Tokyo (JP)

(21) Appl. No.: 18/561,702

(22) PCT Filed: May 12, 2022
(86) PCT No.: PCT/JP2022/020104

§ 371 Date: Nov. 16, 2023

(30) Foreign Application Priority Data
May 18, 2021 (JP) 2021-084023

(51) Int. Cl. A61K 49/18; A61B 5/00; A61B 5/0515

(52) U.S. Cl. CPC A61K 49/186; A61B 5/0042; A61B 5/0515

(57) ABSTRACT

The purpose of the present invention is to provide a contrast medium using a paramagnetic metal that accumulates at an abnormal protein deposition or aggregation site in the brain. Magnetic nanoparticles for image diagnosis which comprise magnetic iron oxide particles, gold fine particles supported on the surface of the magnetic iron oxide particles, a polymer chain attached to the gold fine particles and a molecule directing for a neurodegenerative disease-related protein and attached to at least a part of the polymer chain, and which are to be administered transnasally. These magnetic nanoparticles for image diagnosis are useful as an active ingredient of a contrast medium that accumulates at an abnormal protein deposition or aggregation site in the brain.

### TECHNICAL FIELD

The present invention relates to a magnetic nanoparticle for image diagnosis and a contrast medium for image diagnosis, and specifically relates to a magnetic nanoparticle for image diagnosis that accumulates at a target site in the brain and a contrast medium for image diagnosis using the same.

### BACKGROUND ART

It is expected that the number of dementia patients will become 8 million in 2030, which is a social problem. Since it is difficult to completely cure dementia after onset, diagnosis and prevention before onset are important. Quantum imaging technologies such as PET and MRI can visualize the state in the brain, and thus play an important role in the study of diagnosis, treatment, and prophylaxis of cranial nerve diseases such as dementia.

PET is used to observe specific molecules involved in brain function or pathology, and MRI is used to observe brain activity or morphology. Neurodegenerative diseases such as Alzheimer's disease (AD) are pathologically characterized by accumulation of abnormal proteins inside and outside cells. It is known that senile plaques in which amyloid beta (AP) is deposited extracellularly and neurofibrillary tangles in which tau protein is aggregated intracellularly occur in Alzheimer's disease. Therefore, PET imaging is known as a method for imaging the distribution of abnormal proteins such as Aβ in the brain (See, for example, Non-Patent Document 1.). As a contrast medium used for amyloid PET, injections of labeled low molecular weight compounds such as [¹⁸F]flutemetamol and [¹¹C]Pittsburgh compound are known.

Meanwhile, in recent years, through nasally administering 2-deoxy-2-[¹⁸IF]fluoroglucose ([¹⁸F]FDG), which is a general-purpose PET probe, to a rat as a model drug, visualization of drug absorption kinetics and pharmacokinetic quantitative evaluation after nasal administration in a living body using PET have been reported for the first time in the world (Non-Patent Document 2). In this report, it is pharmacokinetically shown that when the viscosity of the administration solution is increased by adding hydroxypropyl cellulose, improvement in nasal absorbability such as an increase in intranasal retention and an increase in systemic circulation blood transferability is observed.

On the other hand, there is a report on the kinetics when nanoparticles are nasally administered to rats (Non-Patent Document 3). This report shows the particle distribution in various organs after nasally administering polystyrene particles of 20 nm, 100 nm, 50 nm, and 1000 nm to rats, and in particular, it is shown that particles having a particle size of 100 nm or more mostly migrate to blood and are delivered to the liver, while particles having any particle size are almost not delivered to the brain.

### PRIOR ART DOCUMENTS

### Non-Patent Documents

Non-Patent Document 1: Rinsho Shinkeigaku (Clin Neurol) 2017:57:479-484
Non-Patent Document 2: Eur. J. Pharm. Biopharm., 9 9, 45-53 (2016)
Non-Patent Document 3: J Drug Target. 9(4): 267-279, 2001.

### SUMMARY OF THE INVENTION

### Problems to be Solved by the Invention

There are several barriers to widespread PET imaging as a convenient diagnostic imaging method for neurological diseases. First, PET apparatuses are very expensive. Furthermore, since PET contrast mediums have a half-life, the expiration date is very short, and it is necessary to deploy a large drug production facility for producing a radioactive drug as an active ingredient near a medical institution. For this reason, a method without such a barrier related to the device and the contrast medium is desired for diagnosis of a neurological disease.

For example, although the blood test of Aβ is known as a screening test for AD, intracerebral image diagnosis is required for medication of a therapeutic agent, and the FDA cannot start treatment only based on the blood test.

In view of this, for diagnosis of a neurological disease, image diagnosis using paramagnetic metal accumulated at a site where abnormal proteins are deposited or aggregated in the brain as a contrast medium is desirable. However, no such contrast medium is present.

Therefore, an object of the present invention is to provide a contrast medium using a paramagnetic metal that accumulates at a site where abnormal proteins are deposited and aggregated in the brain.

### Means for Solving the Problem

As a result of intensive studies, the present inventor has unexpectedly found that a magnetic nanoparticle containing a magnetic iron oxide particle, a gold fine particle supported on the surface thereof, and a directed molecule for a neurodegenerative disease-related protein bonded to the gold fine particle via a polymer chain accumulates at a site where abnormal proteins are deposited or aggregated in the brain through nasal administration, and enables imaging. Hitherto, in view of the fact that there is no knowledge that a large object such as a nanoparticle reaches the brain as a particle, and furthermore, it has been confirmed that a nanoparticle is not substantially delivered to the brain even if the nanoparticle is nasally administered as shown in Non-Patent Document 3, the effect that the magnetic nanoparticle having a specific configuration reaches the brain through nasal administration is extremely specific. The present invention has been completed by further conducting studies based on this finding.

That is, the present invention provides inventions of the following aspects.
Item 1. A magnetic nanoparticle for image diagnosis, including: a magnetic iron oxide particle; a gold fine particle supported on a surface of the magnetic iron oxide particle; a polymer chain bonded to the gold fine particle; and a directed group for a neurodegenerative disease-related protein bonded to at least a part of the polymer chain, and being nasally administered.
Item 2. The magnetic nanoparticle for image diagnosis according to item 1, wherein the directed group for a neurodegenerative disease-related protein is bonded to 1 to 50 mol % of the polymer chain.
Item 3. The magnetic nanoparticle for image diagnosis according to item 1 or 2, wherein the neurodegenerative disease-related protein is amyloid β.
Item 4. The magnetic nanoparticle for image diagnosis according to any of items 1 to 3, wherein the polymer chain has a weight average molecular weight of 2,000 to 300,000.
Item 5. The magnetic nanoparticle for image diagnosis according to any of items 1 to 4, having an average hydrodynamic diameter of 50 to 200 nm.
Item 6. The magnetic nanoparticle for image diagnosis according to any of items 1 to 5, wherein the polymer chain is polyethylene glycol.
Item 7. A contrast medium for image diagnosis including the magnetic nanoparticle for image diagnosis according to any of items 1 to 6.

### Advantages of the Invention

According to the present invention, it is possible to perform imaging using a paramagnetic metal that accumulates at a site where abnormal proteins are deposited or aggregated in the brain as a contrast medium.

### BRIEF DESCRIPTION OF THE DRAWINGS

FIG. 1 is a TEM image of ABC595-PEG-Au/FcM, a magnetic nanoparticle for image diagnosis prepared in Examples.

FIG. 2 shows a result of anti-PEG staining in Ex vivo experiments that shows binding between ABC595-PEG-Au/FcM, a magnetic nanoparticle for image diagnosis prepared in Examples, and senile plaques in a brain slice.

FIG. 3 shows a result of anti-PEG staining in nasal administration experiments showing that ABC595-PEG-Au/FcM, a magnetic nanoparticle for image diagnosis prepared in Examples, migrated to the brain and bound to senile plaques.

FIG. 4 shows a result of BB staining in nasal administration experiments showing that ABC595-PEG-Au/FcM, a magnetic nanoparticle for image diagnosis prepared in Examples, migrated to the brain and bound to senile plaques.

### EMBODIMENTS OF THE INVENTION

### 1. Magnetic Nanoparticle for Image Diagnosis

The magnetic nanoparticle for image diagnosis of the present invention is characterized by including: a magnetic iron oxide particle; a gold fine particle supported on a surface of the magnetic iron oxide particle; a polymer chain bonded to the gold fine particle; and a directed group for a neurodegenerative disease-related protein bonded to at least a part of the polymer chain, and being nasally administered.

### 1-1. Magnetic Iron Oxide Particle

The magnetic iron oxide particle is an iron oxide fine particle having magnetism. Examples of the iron oxide include FeO, Fe₂O₃, and/or Fe₃O₄, and more specific examples include magnetite (Fe₃O₄), gamma hematite (γ-Fe₂O₃), ferumoxydes ((Fe₂O₃)ₘ(FeO)ₙ (provided that 0<n/m<1)), and ferocarbotran (γ-Fe₂O₃/C₆H₁₁O₆—(C₆H₁₀O₅)ₙ—C₆H₁₁O₅; superparamagnetic iron oxide coated with carboxydextran).

The magnetic iron oxide particle constituting the magnetic nanoparticle for image diagnosis of the present invention has an average particle size as a primary particle of, for example, 2 to 10 nm, preferably 3 to 8 nm, and more preferably 4 to 6 nm. Here, the average particle size as a primary particle in the present invention is a value obtained by randomly selecting 100 primary particles, measuring the diameters of the individual primary particles by transmission electron microscope (TEM) observation, and averaging the diameters. The magnetic iron oxide particle that is a raw material of the magnetic nanoparticle for image diagnosis of the present invention has an average particle size as a secondary particle of, for example, 20 to 100 nm, preferably 40 to 80 nm, and more preferably 50 to 60 nm. Here, the average particle size as a secondary particle in the present invention is a Z-average particle size measured by dynamic light scattering (DLS).

Methods for producing the magnetic iron oxide particle are well known, and specific examples thereof include a physical vapor synthesis (PVS) method (See C. J. Parker, M. N. Ali, B. B. Lympany, U.S. Pat. No. 5,514,349 A.). As the magnetic iron oxide particle, commercially available products can also be used. Preferable commercially available products include Feridex (Registered Trademark; EIKEN CHEMICAL CO. LTD., common name: ferumoxydes, and a colloidal solution of superparamagnetic iron oxide) and Resovist (Registered Trademark; Bayer Yakuhin, Ltd., common name: ferucarbotran, and a hydrophilic colloidal liquid of superparamagnetic iron oxide coated with carboxydextran). Furthermore, when Resovist is used, it is preferable to use particles obtained by magnetically separating ferucarbotran (refer to “Magnetics Japan Vol. 13, No. 4, 2018, Takashi Yoshida, Characterization and Application to Imaging of Magnetic Nanoparticles”).

### 1-2. Gold Fine Particle

In the magnetic nanoparticle for image diagnosis of the present invention, the gold fine particle is supported on the surface of the magnetic iron oxide particle. Preferably, a plurality of gold fine particles is supported on the surface of the magnetic iron oxide particle.

The gold fine particle has an average particle size as a primary particle of, for example, 2 to 10 nm, preferably 3 to 8 nm, and more preferably 4 to 6 nm. The ratio of the average particle size as a primary particle of the gold fine particle to the average particle size as a primary particle of the magnetic iron oxide particle is, for example, 0.4 to 1.4, more preferably 0.7 to 1.2, and further preferably 0.9 to 1.1. In addition, the supported amount of the gold fine particle is, for example, 0.4 to 1.3 parts by weight, preferably 0.6 to 1.2 parts by weight, more preferably 0.8 to 1 part by weight, and still more preferably 0.9 to 0.95 parts by weight as the total weight of the gold fine particle (Au) with respect to 1 part by weight of iron (Fe) in the magnetic iron oxide particle.

As the gold fine particle, a mixture of a gold ion-containing liquid or a gold complex-containing liquid and the magnetic iron oxide particle is irradiated with appropriate energy to cause a reduction reaction of gold ions so that the gold ions are reduced on the surface of the magnetic iron oxide particle and the gold fine particle is formed as a metal particle supported on the surface.

Examples of the solvent of the gold ion-containing liquid and the gold complex-containing liquid include water, alcohol, and mixed solvents thereof. Examples of the alcohol include lower alcohols such as methanol, ethanol, and n-propanol. The gold ion-containing liquid can be prepared by dissolving a compound that gives gold ions in the solvent. Examples of the compound that gives gold ions include gold nitrate, chloride, acetate, and citrate, and gold chloride (HAuCl₄) is preferable. The gold complex-containing liquid can be prepared by dissolving a compound in which an appropriate ligand is coordinated to a gold ion in the solvent. The ligand is not particularly limited as long as it has an unshared electron pair or a negative charge, and examples thereof include a monodentate ligand such as a halide ion, a cyanide ion, ammonia, and pyridine; a bidentate ligand such as ethylenediamine and an acetylacetone ion; and a hexadentate ligand such as an ethylenediaminetetraacetic acid ion. Furthermore, the gold ion-containing liquid and the gold complex-containing liquid can further contain polyvinyl alcohol. The weight average molecular weight of the polyvinyl alcohol is, for example, 10000 to 50000, preferably 15000 to 30000, and more preferably 20000 to 25000. The weight average molecular weight is a weight average molecular weight measured by GPC-LALLS method.

The addition amount of the magnetic iron oxide particle in the mixture is for example, 0.01 to 0.3 g/L, preferably 0.03 to 0.2 g/L, and more preferably 0.05 to 0.15 g/L. In the mixture, the addition amount of gold ions per 1 g of the magnetic iron oxide particle is, for example, 0.5 to 15 mmol, preferably 3 to 10 mmol, and more preferably 4 to 7 mmol. The addition amount of gold ions (Au) to 1 part by weight of iron (Fe) in the iron oxide particle is, for example, 0.4 to 1.3 parts by weight, preferably 0.6 to 1.2 parts by weight, and more preferably 0.8 to 1 part by weight. In the mixture, the addition amount of polyvinyl alcohol per 1 g of the magnetic iron oxide particle is, for example, 50 to 150 g, preferably 80 to 120 g.

Examples of the energy irradiated for causing the reduction reaction of gold ions include gamma ray, electron beam, and ultrasonic wave.

As the gamma ray, a general gamma ray can be used, and a gamma ray derived from cobalt 60 is preferable. The irradiation amount and irradiation time of gamma ray are, for example, about 2 to 4 hours at 2 to 4 kGy/h, and the total irradiation amount is preferably 5 to 15 kGy. As the electron beam, a general electron beam can be used, and an electron beam generated by a linear accelerator is preferable. Examples of the energy of the electron beam include 2 to 15 MeV, preferably 3 to 10 MeV, and more preferably 4 to 6 MeV, and examples of the total surface dose include 1 to 15 kGy, preferably 3 to 10 kGy, and more preferably 5 to 7 kGy. As the ultrasonic wave, a general ultrasonic wave can be used, and an ultrasonic wave of about 150 to 250 kHz is preferable. The irradiation amount and the irradiation time are, for example, about 20 to 40 minutes at 150 to 250 W.

### 1-3. Polymer Chain

The polymer chain is provided using the gold fine particle as a scaffold. The bond between the gold particle and the polymer chain is not particularly limited. Examples thereof preferably include a bond using a bond between a gold atom constituting the gold particle and a sulfur atom, that is, a bond via a sulfur atom. The bond via a sulfur atom is only required to contain at least a sulfide bond. Specific examples thereof include a sulfide bond, a disulfide bond, and a bond in which sulfur and an atom other than sulfur are interposed. Among them, a sulfide bond is preferable as the bond between the gold particle and the polymer chain.

The type of the polymer chain is not particularly limited. Examples thereof include polyalkylene glycol and polyvinyl alcohol, and particularly preferably include polyalkylene glycol. Examples of the polyalkylene glycol include polymethylene glycol, polyethylene glycol, and polypropylene glycol, and polyethylene glycol is preferable.

The length of the polymer chain is, for example, such a length that the weight average molecular weight of the polymer chain is 2000 to 300000, preferably 3000 to 100000, more preferably 4000 to 10000, and still more preferably 4500 to 7000.

The method for bonding the polymer chain to the gold fine particle can be performed by reacting a polymer molecule having a functional group capable of bonding to gold (hereinafter, the group is also described as a “gold-bonding group”) and the polymer chain with the magnetic iron oxide particle having the gold fine particle supported on the surface. In addition, at least a part of the polymer molecule to be reacted with the magnetic iron oxide particle has a functional group to be bonded to a directed molecule for a neurodegenerative disease-related protein described later (hereinafter, the group is also referred to as a “directed molecule-bonding group”), in addition to the gold-bonding group.

That is, when the polymer chain is bonded to the gold fine particle, as the polymer molecule, a polymer molecule having at least both a gold-bonding group and a directed molecule-bonding group (hereinafter, also referred to as “polymer molecule 1”) is used, and preferably, the polymer molecule 1 and a polymer molecule having only a gold-bonding group (hereinafter, also referred to as “polymer molecule 2”) can be used in combination. When the polymer molecule 1 and the polymer molecule 2 are used in combination, the ratio of the polymer molecule 1 when the total amount of the polymer molecule 1 and the polymer molecule 2 is 100 mol % is, for example, 1 to 50 mol %, preferably 3 to 20 mol %, more preferably 5 to 15 mol %, and further preferably 8 to 12 mol %.

The gold-bonding group is not particularly limited as long as it is a functional group having a property of bonding to gold, and preferably includes a thiol group. In addition, the directed molecule-bonding group can be appropriately selected by those skilled in the art according to the functional group the later-described directed molecule has. Preferably, as the directed molecule-bonding group, it is possible to select a functional group having a property of bonding to the functional group the later-described directed molecule has and different from the gold-bonding group.

In the polymer molecule 1 and the polymer molecule 2, the site where the gold-bonding group is bonded is not particularly limited, and preferably includes the end of the polymer chain. In the polymer molecule 1, the site where the directed molecule-bonding group is bonded is not particularly limited, and preferably includes a site farthest from the site where the gold-bonding group is bonded, and more preferably, when the gold-bonding group is bonded to one end of the polymer chain, the other end.

The reaction conditions for bonding the polymer molecule to the gold fine particle can be appropriately determined by those skilled in the art depending on the type of the gold-bonding group. Specifically, it is about 16 to 25° C. for about 30 to 1.5 hours.

### 1-4. Directed Group for Neurodegenerative Disease-Related Protein

The directed group for a neurodegenerative disease-related protein is bonded to at least a part of the polymer chain. Specifically, the directed group for a neurodegenerative disease-related protein is bonded to the polymer chain derived from the polymer molecule 1 via the directed molecule-bonding group. The mode of bonding via the directed molecule-bonding group may be a covalent bond or a non-covalent bond, but is preferably a covalent bond.

The bonding amount of the directed group for a neurodegenerative disease-related protein is not particularly limited. When the total amount of the polymer chain is 100 mol %, the bonded proportion of the directed group is, for example, 1 to 50 mol %, preferably 3 to 20 mol %, more preferably 5 to 15 mol %, and further preferably 8 to 12 mol %.

The neurodegenerative disease is not particularly limited, and examples thereof include Alzheimer's disease, Alzheimer's disease, dementia with Lewy bodies, Parkinson's disease, amyotrophic lateral sclerosis, and frontal lobe temporal lobe degeneration. The neurodegenerative disease-related protein is not particularly limited as long as it is an abnormal protein that is deposited and/or aggregated in the neurodegenerative disease, and examples thereof include amyloid β (Aβ), tau, and α synuclein. The directed molecule that provides the directed group for a neurodegenerative disease-related protein (That is, a molecule containing the directed group and the above-described directed molecule-bonding group) is not particularly limited as long as it is a molecule that specifically bonds to the neurodegenerative disease-related protein. For example, among the directed groups, examples of the Aβ directed group include the structures (moieties other than the R group) shown in (i) to (vi).

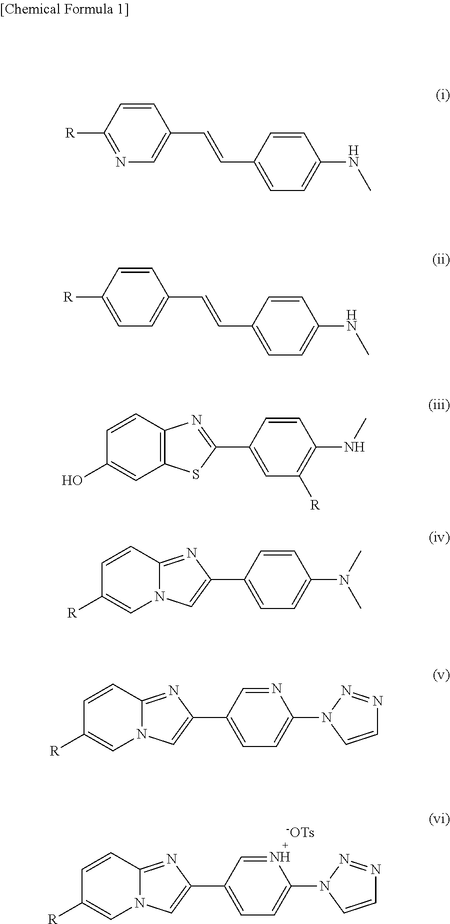

The directed group shown in (i) is a group [¹⁸F]florbetapir has. The directed group shown in (ii) is a group [¹⁸F]florbetaben has. The directed group shown in (iii) is a group [¹⁸F]flutemetamol has. The directed group shown in (iv) is a group [¹²³I]IMPY has. The directed group shown in (v) is a group ABC577, ABC594, and ABC595 have. The directed group shown in (vi) is a group ABC595 has.

The directed group is directly or indirectly (that is, via a linking group) bonded to the polymer chain. Specific examples of the linking group include any divalent group, and include an ester group (—COO—), an ether group (—O—), an amide group (—NHCO—), a carbonyl group (—CO—), an oligoalkylene glycol group (Examples thereof include those having a molecular weight of 100 to less than 2000, preferably 150 to 1000 or less, and more preferably 100 to 200 or less.), and a group obtained by combining a plurality of groups from these groups.

The method for bonding the directed molecule for a neurodegenerative disease-related protein to the polymer chain derived from the polymer molecule 1 can be appropriately determined by those skilled in the art according to the type of the directed molecule-bonding group and the directed group of the directed molecule corresponding thereto.

### 1-5. Particle Size

The particle size of the magnetic nanoparticle for image diagnosis of the present invention is not particularly limited as long as the particle can be transferred into the brain by nasal administration. The average hydrodynamic diameter is preferably 50 to 20 nm, preferably 50 to 150 nm, more preferably 55 to 120 nm, still more preferably 60 to 110 nm, still more preferably 80 to 110 nm, and still more preferably 95 to 110 nm. The average hydrodynamic diameter means Z-average hydrodynamic diameter measured by dynamic light scattering (DLS).

### 1-6. Application

The magnetic nanoparticle for image diagnosis of the present invention is used as a magnetic nanoparticle for image diagnosis to be nasally administered. That is, the magnetic nanoparticle for image diagnosis of the present invention is used as an active ingredient of a contrast medium for nasal administration for performing image diagnosis using magnetism. Examples of the image diagnosis using magnetism usually include magnetic resonance imaging (MRI), magnetic particle imaging (MPI), and the like.

### 1-7. Dose

The dose of the magnetic nanoparticle for image diagnosis of the present invention is not particularly limited on the condition that it is possible to migrate to the brain to bind to the abnormal proteins in the brain after nasal administration. For example, the dose to human can be appropriately determined within a range of 0.1 to 10 mg/kg, preferably 0.3 to 7 mg/kg in consideration of the maximum safe dose of the magnetic iron oxide particle and the like.

### 2. Contrast Medium for Image Diagnosis

As described above, the magnetic nanoparticle for image diagnosis of the present invention is used as an active ingredient of a contrast medium for nasal administration for performing image diagnosis using magnetism. Therefore, the present invention also provides a contrast medium for image diagnosis including the above-described magnetic nanoparticle for image diagnosis.

The contrast medium for image diagnosis of the present invention is formulated as a nasal administration agent by a known means using the magnetic nanoparticle for image diagnosis as an active ingredient, and a pharmacologically acceptable base and/or additive may be appropriately mixed in addition to the active ingredient.

Examples of the pharmacologically acceptable base and/or additive include water, an organic solvent, an excipient, a mucous membrane absorption promoter (such as sodium decanoate), a thickening agent (hydroxypropyl methyl cellulose (HPMC), carboxymethyl cellulose (CMC), hydroxyethyl cellulose (HEC), carbopol, methyl cellulose (MC), and the like), a lubricant, a binder, a disintegrant, a solubilizing agent, a suspending agent, an emulsifier, an isotonizing agent, a buffering agent, a soothing agent, a stabilizer, a preservative (antiseptics), a pH adjusting agent, a cooling agent, an antioxidant, a wetting agent, and a flavoring agent.

The form of the contrast medium for image diagnosis of the present invention may be either a liquid agent or a solid agent, but a liquid agent is preferable. When the contrast medium for image diagnosis of the present invention is prepared as a liquid preparation, for example, the contrast medium can be produced by mixing an active ingredient with a solvent, a mucous membrane absorption promoter, a solubilizing agent, a suspending agent, an isotonizing agent, a buffering agent, a soothing agent, and/or the like to be blended as necessary, and dissolving, suspending, or emulsifying the mixture, and if necessary, by further adding a thickening agent, the viscosity can be increased, and retention can be imparted. When the contrast medium for image diagnosis of the present invention is prepared as a solid agent, the contrast medium can be produced, for example, by uniformly mixing an active ingredient with a mucous membrane absorption promoter, an excipient, a binder, a disintegrant, and/or the like to be blended as necessary, obtaining a granulated product by an appropriate granulation method, and further forming the granulated product into a powder or fine particle by drying as necessary.

The contrast medium for image diagnosis of the present invention can be used by being filled in a container for nasal administration. As the container for nasal administration, a commercially available container can be used as appropriate.

### EXAMPLES

Hereinafter, the present invention will be described more specifically with reference to examples, but the present invention is not limited thereto.

### [Test Example 1] Preparation of Magnetic Nanoparticle for Image Diagnosis

### (1) Support of Gold Fine Particle on Surface of Iron Oxide Nanoparticle

A particle dispersion (iron concentration: 56 mg/ml) obtained by magnetically separating fercarbotolane, which is a raw material of Resovist (registered trademark), by a permanent magnet was prepared. Specifically, first, as an aqueous solution in which raw materials were dispersed, an aqueous solution containing ultrapure water, iron oxide nanoparticle (ferocarbotran, final concentration 0.1 g/L), gold chloride (HAuCl₄, final concentration 0.5 mmol/L), 2-propanol (final concentration: 10 mL/L), and polyvinyl alcohol (weight average molecular weight: 22000, final concentration: 10 g/L) was prepared. Next, 50 mL of the raw material aqueous solution was sealed in a glass vial, and irradiated with an accelerator electron beam. The irradiation was performed at an acceleration voltage of 4.8 MeV until the surface dose reached 6 kGy. As a result, gold ions in the raw material aqueous solution were reduced to gold fine particles by the chemical reaction proceeding by radiation irradiation, and gold fine particles were generated in a state of being supported on the iron oxide surface.

About 15 ml of the irradiated solution was put into an ultrafiltration filter (Amicon Ultra-15, molecular weight cutoff: 1,000 kDa, Merck), and centrifugation was performed at 5000 G for 10 minutes to remove impurities and simultaneously remove water, thereby purifying and concentrating the particle. As a result, a dispersion of an iron oxide nanoparticle supporting gold nanoparticles (gold/iron oxide nanoparticle) was obtained.

### (2) Polyethylene Glycol (PEG) Modification of Gold/Iron Oxide Nanoparticle

As the polymer molecule 1, a molecule having a thiol group (gold-bonding group) on one end of a PEG chain and an amino group (directed molecule-bonding group) on the other end (SH-PEG-NH₂; manufactured by Creative PEG Works) was prepared. As the polymer molecule 2, a molecule having only a thiol group (gold-bonding group) on one end of a PEG chain (PEG-SH: manufactured by SunBright) was prepared. Both the polymer molecule 1 and the polymer molecule 2 have a molecular weight of 5000 Da. A solution containing both the polymers in such a ratio that the polymer molecule 1 accounts for 10 mol % based on 100 mol % of the total amount of the polymer molecule 1 and the polymer molecule 2 was prepared as a reagent solution for modification.

The gold/iron oxide nanoparticle and the reagent solution for modification were mixed and stirred for 2 hours in such a ratio that PEG was 10 parts by weight based on 1 part by weight of gold to specifically bond the thiol groups on the end of the polymer molecule 1 and the polymer molecule 2 to the gold atoms on the surface of the gold fine particle, thereby immobilizing the PEG chains to the surface of the iron oxide nanoparticle using the gold fine particles as a scaffold. Thereafter, the reaction solution was sealed in a 50 ml dialysis tube, and dialyzed in 2 L of ultrapure water for 24 hours using a dialysis membrane (Spectra/pore CE, molecular weight cutoff: 1,000 kDa, manufactured by Funakoshi) to obtain a sample solution from which the excessive polymer molecule 1 and polymer molecule 2 had been removed.

A magnetic separation column (25 MS Columns: manufactured by Miltenyi Biotec) was set in a dedicated permanent magnet adapter, the sample solution after dialysis was charged, nonmagnetic components were completely removed to the outside of the column, pure water in the same amount as the sample solution was then charged, and gold physically remaining in the column was washed away. Thereafter, the column was removed from the adapter, and pure water was charged into the column and extruded with a syringe to recover the magnetic component in the column. The recovered liquid was subjected to a freeze-drying treatment to obtain a PEG-modified magnetic nanoparticle powder.

### (3) Immobilization of Amyloid β-Directed Molecule on PEG Chain

As a directed molecule for amyloid β, ABC595, which is shown in the following formula, was used. The ABC595 molecule has an NHS (N-hydroxysuccinimide) group as a functional group corresponding to the amino group (directed molecule-bonding group) derived from the polymer molecule 1, and a group shown in the above formula (v) as a directed group.

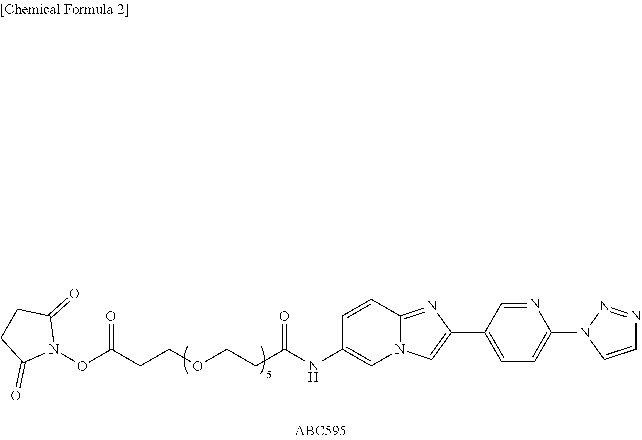

A dimethylformamide solution of ABC595 and a dispersion in which the PEG-modified magnetic nanoparticle was dispersed in a PBBS buffer solution (adjusted to pH=7.4, manufactured by Sigma-Aldrich Co. LLC) were mixed in such a ratio that the ABC595 molecule was 0.5 parts by weight based on 1 part by weight of Fe of the magnetic nanoparticle, and the mixture was stirred at 4° C. for 3 hours to react the amino group (directed molecule-bonding group) present on a part of the PEG chain on the surface of the magnetic nanoparticle with the NHS group of the ABC595 molecule, so that the directed group of the ABC595 molecule was immobilized.

Thereafter, the reaction solution was sealed in a 50 ml dialysis tube, and a dialysis membrane (Spectra/pore CE, molecular weight cutoff: 1,000 kDa, manufactured by Funakoshi) was used to perform a dialysis treatment in 2 L of ultrapure water for 24 hours. The sample after the dialysis treatment was subjected to a freeze-drying treatment to obtain a powder of ABC595-PEG-Au/FcM (magnetic nanoparticle for image diagnosis), a magnetic nanoparticle in which the directed group of ABC595 was bonded (immobilized) to 10 mol % of all the surface PEG chains.

### (4) Evaluation of Physical Properties of Particle

### (4-1) Measurement of Particle Size

### (TEM Observation)

The morphology of the particle was observed using a transmission electron microscope (TEM: Transmission Electron Microscope, JEM-2100, which is manufactured by JEOL Ltd.) to determine the average particle size of the primary particle. Specifically, about 1 to 2 drops of the particle dispersed by ultrasonic cleaning were dropped with a dropper on a copper mesh (manufactured by NISSHIN EM Co. Ltd.) covered with a carbon film, and then dried in a dryer at about 60° C. for 24 hours or more to prepare an observation sample. One hundred primary particles were randomly selected, and the diameter of each of the primary particles was measured by TEM observation and averaged.

### (DLS Measurement)

The particle size of an aggregate formed by weakly aggregating primary particles in a fluid is referred to as a secondary particle size. The particle was sufficiently and uniformly dispersed with ultrasonic wave using a dynamic light scattering (DLS) apparatus (ZETASIZER NANO-ZS; manufactured by Spectris Co., Ltd.), and the average particle size of the secondary particle was measured as Z average particle size.

The iron oxide nanoparticle (ferucarbotran) magnetically separated with a permanent magnet had an average particle size as a primary particle size of 5 mm and an average particle size as a secondary particle of 54 nm.

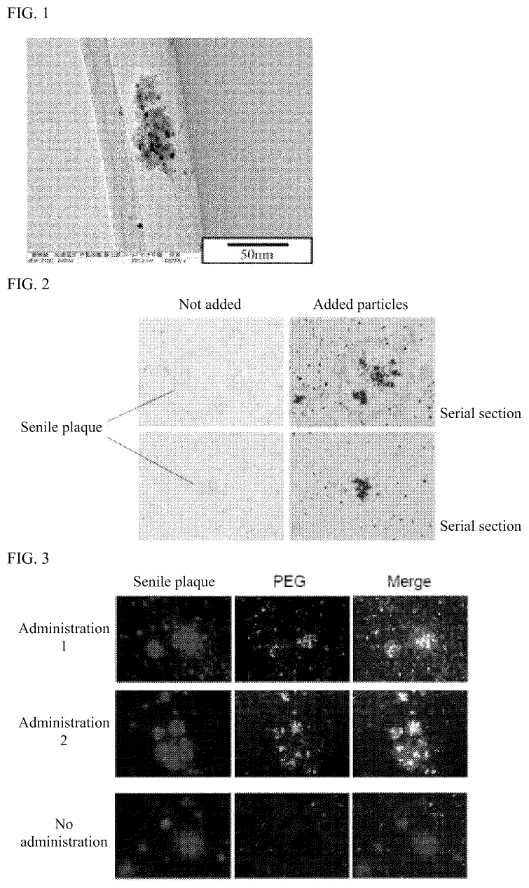

The TEM observation result of the magnetic nanoparticle for image diagnosis ABC595 directed group-PEG-Au/FcM is shown in FIG. 1. In FIG. 1, it was confirmed that the particles having a weak contrast were iron oxide nanoparticles and the strong contrast were gold nanoparticles, and both the particles formed a weak aggregate. The average particle size of the iron oxide nanoparticle in the ABC595 directed group-PEG-Au/FcM was 5 nm, which was almost the same as that of the raw material. The average particle size of the gold nanoparticle was also about 5 nm. The average particle size as a secondary particle of the ABC595 directed group-PEG-Au/FcM in water determined by DLS measurement was 108 nm. Furthermore, the weight ratio of iron (Fe) and gold (Au) determined by ICP analysis was 1:0.92.

Incidentally, a magnetic field magnetization curve between the ABC595 directed group-PEG-Au/FcM and the iron oxide nanoparticle as a raw material was obtained. It was confirmed that when the magnetic field magnetization curve was expressed by normalizing the magnetization value on the vertical axis with the saturation magnetization of each particle, both lines were almost completely overlapped, that is, the magnetization behaviors of both the particles were almost the same. From this fact, it was acknowledged that the process of PEG modification and immobilization of the probe molecule ABC595 (directed molecule) maintained the magnetic properties of the raw material iron oxide nanoparticle without any change.

### (5) Ex-Vivo Studies

The ABC595 directed group-PEG-Au/FcM was dispersed in ultrapure water, subjected to ultrasonic treatment, and dispersed in a tris buffer containing 0.3% triton X100 and 20% calf serum so that the ABC595 directed group-PEG-Au/FcM had a concentration of 25 μg/mL, thereby preparing a contrast medium for image diagnosis. The contrast medium for image diagnosis was dropped on a brain cell slice of a model mouse containing cells (senile plaques) in which amyloid β accumulated. As the brain slice, one obtained by boiling in a pH 2 hydrochloric acid solution and suppressing the non-specific reaction with a 20% calf serum-containing tris buffer was used. The contrast medium for image diagnosis was reacted with the brain slice at 37° C. for 3 hours, and then washed using a tris buffer solution, and an anti-PEG mouse monoclonal antibody was added dropwise as a primary antibody and reacted overnight at 4° C. After the primary antibody reaction, washing was performed, and a biotinylated anti-mouse Ig antibody as a secondary antibody was added dropwise and reacted at room temperature for 1 hour. After the secondary antibody reaction, washing, sensitization by the avidin-biotin complex method, and an addition of peroxidase were performed. After washing, the brain slice was immersed in a DAB solution as a substrate, and the reaction site of the contrast medium for image diagnosis was caused to develop color by peroxidase activity.

The staining results (n=2) of the brain slices before the contrast medium was reacted (not added) and after the contrast medium was reacted (added particles) are shown in FIG. 2. As is clear from FIG. 2, it was confirmed that the magnetic nanoparticles of the contrast medium bound to the senile plaques on the brain slice.

### [Test Example 2] Intracerebral Imaging by Nasal Administration of Contrast Medium for Image Diagnosis

The ABC595 directed group-PEG-Au/FcM was dispersed in ultrapure water, subjected to ultrasonic treatment, and dispersed in an aqueous solution containing 0.5 wt % sodium decanoate and 0.25 wt % carboxymethylcellulose so that the ABC595 directed group-PEG-Au/FcM had a concentration of 10 mg/mL, thereby preparing a contrast medium for image diagnosis. Into the nasal cavity of an AD model mouse, 20 μL of the contrast medium for image diagnosis was administered, and after 6 hours, reflux fixation was performed to prepare a brain slice. The brain slice was boiled in a pH 2 hydrochloric acid solution, and then a non-specific reaction was suppressed by a 20% calf serum-containing tris buffer solution. A mixed solution of an anti-PEG mouse monoclonal antibody and an anti-amyloid β rabbit polyclonal antibody was added dropwise as a primary antibody, and reacted overnight at 4° C. After the primary antibody reaction, washing was performed, and a mixed solution of a FITC-conjugated anti-mouse Ig antibody and a Rhodamin-conjugated anti-rabbit Ig antibody was added dropwise as a secondary antibody to cause a reaction at room temperature for 1 hour.

The result of anti-PEG staining (n=2) of the brain slice of the mouse to which the contrast medium was nasally administered is shown in FIG. 3. As is clear from FIG. 3, it was observed under a fluorescence microscope that the contrast medium for image diagnosis indicated by FITC was bound to senile plaques indicated by Rhodamin. That is, it was confirmed that the magnetic nanoparticles of the contrast medium administered nasally were transferred to the brain and bound to senile plaques.

In addition, the brain slices boiled in a pH 2 hydrochloric acid solution were caused to develop color in a mixed solution of equal amount of 2% potassium ferrocyanide and 2% hydrochloric acid, and were also used for detection of iron particles by Berlin blue (BB) staining.

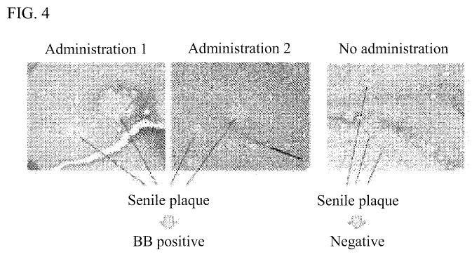

The BB staining result (n=2) of the brain slice of the mouse to which the contrast medium was nasally administered is shown in FIG. 4. As is clear from FIG. 4, it was confirmed that the magnetic nanoparticles of the contrast medium administered nasally migrated to the brain and bound to senile plaques.

1. A magnetic nanoparticle for image diagnosis, comprising:
a magnetic iron oxide particle;
a gold fine particle supported on a surface of the magnetic iron oxide particle;
a polymer chain bonded to the gold fine particle; and
a directed group for a neurodegenerative disease-related protein bonded to at least a part of the polymer chain.

2. The magnetic nanoparticle for image diagnosis according to claim 1, wherein the directed group for a neurodegenerative disease-related protein is bonded to 1 to 50 mol % of the polymer chain.

3. The magnetic nanoparticle for image diagnosis according to claim 1, wherein the neurodegenerative disease-related protein is amyloid β.

4. The magnetic nanoparticle for image diagnosis according to claim 1, wherein the polymer chain has a weight average molecular weight of 2,000 to 300,000.

5. The magnetic nanoparticle for image diagnosis according to claim 1, having an average hydrodynamic diameter of 50 to 200 nm.

6. The magnetic nanoparticle for image diagnosis according to claim 1, wherein the polymer chain is polyethylene glycol.

7. A contrast medium for image diagnosis comprising the magnetic nanoparticle for image diagnosis according to claim 1.

8. A method of treating a subject comprising administering the magnetic nanoparticle according to claim 1 to the subject.

9. The method according to claim 8, wherein the magnetic nanoparticle is nasally administered.

10. The method according to claim 1, wherein the subject suffers from a neurodegenerative disease.
